(19) United States
(12) Patent Application Publication
(10) Pub. No.: US 2024/0269279 A1
(43) Pub. Date: Aug. 15, 2024

(54) PHARMACEUTICAL FORMULATION CONTAINING AN ANTI-IgE ANTIBODY

(71) Applicant: NOVARTIS AG, Basel (CH)

(72) Inventors: Ivan BOTTOLLI, Basel (CH); Andreas FISCH, Zug (CH)

(21) Appl. No.: 18/569,346

(22) PCT Filed: Jun. 14, 2022
(86) PCT No.: PCT/IB2022/055485

§ 371 Date: Dec. 12, 2023

(30) Foreign Application Priority Data
Jun. 14, 2021 (WO) PCT/IB2021/055217

(51) Int. Cl. A61K 39/395; A61K 9/08; A61K 39/00; A61K 47/18; A61K 47/22; A61K 47/26; C07K 16/42

(52) U.S. Cl. CPC A61K 39/39566; A61K 9/08; A61K 39/39591; A61K 47/183; A61K 47/22; A61K 47/26; C07K 16/4291; A61K 2039/505; C07K 2317/94

(57) ABSTRACT

The present invention provides anti-lgE antibodies formulated as stable aqueous pharmaceutical compositions. suitable for injection. An aqueous pharmaceutical composition of the invention includes a sugar (trehalose). a buffering agent (histidine). and a surfactant (polysorbate 20). The aqueous pharmaceutical compositions are useful for delivery of a high concentration of the antibody (at least 50 mg/ml) active ingredient to a patient without high levels of antibody aggregation and without a high level of sub-visible particulate matter.

### FIELD OF THE INVENTION

The present invention relates to aqueous pharmaceutical formulations of anti-IgE antibodies, such as ligelizumab, a process for the preparation thereof, and uses of the formulations.

### BACKGROUND OF THE INVENTION

The present invention relates to novel pharmaceutical formulations, in particular novel pharmaceutical formulations in which the active ingredient comprises antibodies to IgE, in particular antibodies described in WO04/70011 and WO05/75504, in particular ligelizumab.

Antibodies, as other protein therapeutics are complex molecules and in general, large amounts of antibodies have to be used in pharmaceutical formulations due to their therapeutically effective dose in mammals, particularly humans. Liquid formulations of protein therapeutics should preserve intact the biologic activity of the protein therapeutics and protect the functional groups of the protein therapeutics from degradation during manufacturing and shelf life. Degradation pathways for proteins can involve chemical instability or physical instability.

A long appreciated problem with liquid formulations of protein therapeutics is that of aggregation, where protein molecules physically stick together, for example, resulting in the formation of opaque insoluble matter or precipitation, which may show undesired immunological reactions. Additionally, a major problem caused by the aggregate formation is that during the administration the formulation may block syringes or pumps and rendering it unsafe to patients. However, formulations with high concentration of antibody may have short shelf lives, and the formulated antibodies may lose biological activity caused by chemical and physical instabilities during storage. Aggregation, deamidation and oxidation are known to be the most common causes of antibody degradation. In particular, aggregation can potentially lead to increased immune response in patients, leading to safety concerns. Thus it must be minimized or prevented.

Methods for producing high concentration antibody formulations are known. However, a universal approach does not exist to overcome the unpredictable impact of an antibody's amino acid sequence on its tendency to form aggregates or degrade in the presence of various pharmaceutical excipients, buffers, etc. Development of formulations for protein drugs requiring high dosing is challenging for solubility limited proteins and also results in several manufacturing, stability, analytical, and delivery challenges. The concentration dependent degradation route of aggregation is the greatest challenge to developing protein formulation. In addition to the potential for non-native protein aggregation and particulate formation, reversible self-association may occur, which contributes to properties such as viscosity that complicates delivery by injection. In addition, aqueous protein formulations may become cloudy and turbid over time as they are stored, for example in a refrigerator or freezer. Cloudiness and turbidity are generally associated with aggregation or crystallization of the proteins in the formulation. There is a strong preference to avoid any cloudiness or turbidity in a protein formulation to avoid any need for filtration or other means of clarifying the formulation before injection or otherwise delivering it to the patient.

Liquid pharmaceutical composition comprising an anti-IgE antibody, suitable for injection, are known from WO2004091658. In WO2004091658 it is disclosed that arginine, specifically arginine-HC1, in an amount of 50-200 mM, may be required for stable, highly concentrated liquid anti-IgE antibody formulations. Exemplified are stable anti-IgE antibody formulations that overcome the challenges of viscosity, osmolarity and turbidity, all comprising the excipient arginine-HCl.

However, it is also known that arginine may interact with aromatic amino acid residues in proteins, such antibodies. Due to the physicochemical differences inherit to individual monoclonal antibodies, providing high concentration formulation, which are stable and with a desired viscosity remains technically challenging.

It is an object of the invention to provide further and improved formulations, e.g. with high concentration, of anti-IgE antibodies and low levels of antibody aggregation, that are stable and suitable for administration to a human, and which avoid cloudiness/turbidity/crystallization.

### SUMMARY OF THE INVENTION

Accordingly, the present invention is directed to an aqueous pharmaceutical composition comprising an anti-IgE antibody, suitable for injection. In certain aspects, the aqueous pharmaceutical compositions of the invention exhibit low to undetectable levels of antibody aggregation or degradation, with very little to no loss of the biological activities during manufacture, preparation, transportation and long periods of storage, the concentration of the anti-IgE antibody being at least about 50 mg/ml, 60 mg/ml, 80 mg/ml, 90 mg/ml, 100 mg/ml, 120 mg/ml, 140 mg/ml, 150 mg/ml, or 160 mg/ml.

The invention provides aqueous pharmaceutical compositions comprising an anti-IgE antibody, a stabilizer, a buffer, and a surfactant. In certain aspects, as aqueous pharmaceutical composition comprises: (i) at least 50 mg/ml of an anti-IgE antibody, (ii) a sugar (such as trehalose) as a stabilizer, (iii) a histidine buffer, and (iv) polysorbate 80 or polysorbate 20 as a surfactant.

In certain aspects, the aqueous pharmaceutical composition comprises at least 120 mg/ml of the anti-IgE antibody ligelizumab, about 10-30 mM histidine buffer, about 200-270 mM trehalose, about 0.01-0.03% polysorbate 20, wherein the pH of the composition is about 4.7 to about 5.2.

In certain aspects, the aqueous pharmaceutical composition comprises at least 120 mg/ ml of the anti-IgE antibody ligelizumab, about 20 mM histidine buffer, about 250 mM trehalose, about 0.02% polysorbate 20, wherein the pH of the composition is about 4.7 to about 5.2.

Specific preferred embodiments of the invention will become evident from the following more detailed description of certain preferred embodiments and the claims.

### DETAILED DESCRIPTION OF THE INVENTION

The invention provides a stable aqueous pharmaceutical compositions comprising an anti-IgE antibody, e.g. a high concentration of an anti-IgE antibody. In certain embodiments an aqueous pharmaceutical composition of the invention is stable for at least 18 months at 2-8° C. and is suitable for administration to a subject in need thereof, including injection or infusion, e.g., subcutaneous administration.

The present invention provides novel pharmaceutical formulations, in particular novel pharmaceutical formulations in which the active ingredient comprises anti-IgE antibody, such as ligelizumab. In one aspect, the invention relates to a stable aqueous pharmaceutical composition with ligelizumab.

In a preferred embodiment, an aqueous pharmaceutical composition of the invention comprises ligelizumab.

In some embodiments, the anti-IgE antibody, such as ligelizumab, may refer to antibodies which have demonstrated to be biosimilar to or interchangeable to ligelizumab. A biosimilar to ligelizumab is a biosimilar product which contains a version of ligelizumab, as defined by, for example, in the Biosimilar Guidances issued by relevant health authorities (e.g. World Health Organization. Expert Committee on Biological Guidelines on evaluation of similar biotherapeutic products (SBPs). Geneva, Switzerland: World Health Organization; 2009). Those antibodies may be formulated according the embodiments which refer to ligelizumab formulation, as herein disclosed.

In one embodiment, the concentration of an anti-IgE antibody, e.g. ligelizumab, in the aqueous pharmaceutical composition of the invention is at least 50 mg/ml. Preferably, the aqueous pharmaceutical composition of the invention comprises about 50 mg/ml, about 60 mg/ml, about 70 mg/ml, about 80 mg/ml, about 90 mg/ml, about 100 mg/ml, about 110 mg/ml, about 120 mg/ml, about 130 mg/ml, about 140 mg/ml, or about 150 mg/ml of the anti-IgE antibody, e.g. ligelizumab.

In certain embodiments, the aqueous pharmaceutical composition of the invention comprises between about 100 mg/ml and about 120 mg/ml of an anti-IgE antibody, for example, ligelizumab.

In certain embodiments, the aqueous pharmaceutical composition of the invention comprises about 120 mg/ml of an anti-IgE antibody, for example, ligelizumab.

The term “antibody” as used herein includes whole antibodies and any antigen binding fragment (i.e., “antigen-binding portion,” “antigen binding polypeptide,” or “immunobinder”) or single chain thereof. An “antibody” includes a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antibody portion”) refers to one or more fragments of an antibody that retain the ability to specifically bind to an antigen (e.g., IgE). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, CL and CH1 domains; (ii) a F(ab′)₂ fragment, a bivalent fragment comprising two Fab fragments linked by a disulfide bridge at the hinge region; (iii) a Fd fragment consisting of the V^{H} and CH1 domains; (iv) a Fv fragment consisting of the V^{L} and V^{H} domains of a single arm of an antibody, (v) a single domain or dAb fragment (Ward et al., (1989) Nature 341:544-546), which consists of a VH domain; and (vi) an isolated complementarity determining region (CDR) or (vii) a combination of two or more isolated CDRs which may optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins. Antibodies can be of different isotype, for example, an IgG (e.g., an IgG1, IgG2, IgG3, or IgG4 subtype), IgA1, IgA2, IgD, IgE, or IgM antibody.

As used herein, the term “about” includes and describes the value or parameter per se. For example, “about x” includes and describes “x” per se. As used herein, the term “about” when used in association with a measurement, or used to modify a value, a unit, a constant, or a range of values, refers to variations of ±1-10% in addition to including the value or parameter per se. In some embodiments, the term “about” when used in association with a measurement, or used to modify a value, a unit, a constant, or a range of values, refers to variations of ±1, ±2, ±3, ±4, ±5, ±6, ±7, ±8, ±9, or ±10%.

As used herein, the term “between” includes and describes the value or parameter per se. For example, “between x and y” includes and describes “x” and “y”.

As used herein, the term “Viscosity” may be “kinematic viscosity” or “absolute viscosity.” “Kinematic viscosity” is a measure of the resistive flow of a fluid under the influence of gravity.

When two fluids of equal volume are placed in identical capillary viscometers and allowed to flow by gravity, a viscous fluid takes longer than a less viscous fluid to flow through the capillary. If one fluid takes 200 seconds to complete its flow and another fluid takes 400 seconds, the second fluid is twice as viscous as the first on a kinematic viscosity scale. “Absolute viscosity”, sometimes called dynamic or simple viscosity, is the product of kinematic viscosity and fluid density. The dimension of kinematic viscosity is L2/T where L is a length and T is a time. Commonly, kinematic viscosity is expressed in centistokes (cSt). The SI unit of kinematic viscosity is mm2/s, which is 1 cSt. Absolute viscosity is expressed in units of centipoise (cP). The SI unit of absolute viscosity is the milliPascal-second (mPa.s), where 1 cP=1 mPa·s.

As used herein, the term “stable” means that the pharmaceutical formulation containing the anti-IgE antibody as described herein essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N. Y., Pubs. (1991) and Jones, A. Adv. Drug Delivery Rev. 10: 29-90 (1993), for example. Stability can be measured at a selected temperature for a selected time period, for example using AEX-HPLC (Anion exchange high performance liquid chromatography) as described herein. Preferably, the aqueous formulation is stable at room temperature (about 25° C.) or at 40° C.for at least 1 week and/or stable at about 2-8° C. for at least 3 months, at least 12 months, at least 18 months, or at least 24 months. As used herein, the term “stable” also means that the formulation containing an anti-IgE antibody, such as ligelizumab, meets the regulatory requirements for pharmaceutical products.

The anti-IgE antibody as described herein “retains its physical stability” in a pharmaceutical formulation if it meets the defined release specifications for aggregation, degradation, precipitation and/or denaturation upon visual examination of color and/or clarity, or as measured by UV light scattering, AEX-HPLC, or by size exclusion chromatography (SEC), or other suitable methods known in the art.

As used herein, the term “protein aggregation” means the formation of protein species of higher molecular weight, such as oligomers or multimers, instead of the desired defined species of the biopharmaceutical drug (e.g., a monomer). Protein aggregation is thus a universal term for the formation of all kinds of not further defined multimeric species that are formed by covalent bonds or noncovalent interactions. Aggregates can be measured by Size Exclusion Chromatography (SE-HPLC or SEC). In one embodiment, aggregates of the anti-IgE antibody in the aqueous pharmaceutical formulation are below the limit of quantitation.

The anti-IgE antibody as described herein “retains its stability” in an aqueous pharmaceutical formulation, if the purity of the antibody does not decrease, or does not substantially decrease, after storage at room temperature (about 25° C.) or at 40° C. for at least 1 week and/or stable at about 2-8° C. for at least 3 months to 18 months. Stability of the anti-IgE antibody may be assessed by any suitable means, for example, size-exclusion chromatography (SEC), capillary gel electrophoresis and/or anion exchange chromatography (AEX). In one embodiment, the anti-IgE antibody is stable in an aqueous pharmaceutical composition, wherein the % loss in main peak assessed by SEC is ≤5%, ≤4%, ≤3%, ≤2%, ≤1%, ≤0.5%, <0.4%, ≤0.3%, <0.2% or ≤0.1% assessed after storage at room temperature (about 25° C.) or at 40° C.for at least 1 week and/or at about 2-8° C. for at least 3 months, at least 6 months, at least 9 months, at least 12 months, or at least 18 months. In a preferred embodiment, the anti-IgE antibody has <0.5%, ≤0.4%, ≤0.3%, ≤0.2% or ≤0.1% loss in main peak assessed by SEC after storage at about 2-8° C. for at least 3 months, at least 6 months, at least 9 months, at least 12 months, or at least 18 months.

In one embodiment, the anti-IgE antibody is stable in an aqueous pharmaceutical composition, wherein the % loss in sum of HC and LC assessed by capillary gel electrophoresis, for example under reducing conditions, e.g., SDS, is ≤5%, ≤4%, ≤3%, ≤2%, ≤1%, ≤0.5%, ≤0.4%, ≤0.3%, or ≤0.2% assessed after storage at room temperature (about 25° C.) or at 40° C. for at least 1 week and/or at about 2-8° C. for at least 3 months, at least 6 months, at least 9 months, at least 12 months, or at least 18 months. In a preferred embodiment, the anti-IgE antibody has ≤0.5%, ≤0.4%, <0.3%, or ≤0.2% loss in sum of HC and LC assessed by capillary gel electrophoresis after storage at about 2-8° C. for at least 3 months, at least 6 months, at least 9 months, at least 12 months, or at least 18 months. In a particularly preferred embodiment, the anti-IgE antibody has <0.2% loss in sum of HC and LC assessed by capillary gel electrophoresis after storage at about 2-8° C. for at least 3 months, at least 6 months, at least 9 months, at least 12 months, or at least 18 months.

In one embodiment, the anti-IgE antibody is stable in an aqueous pharmaceutical composition, wherein the % sum of acidic peaks assessed by anion exchange chromatography (AEX) is ≤2%, ≤1.9%, ≤1.8%, ≤1.7%, or ≤1.6% assessed after storage at about 2-8° C. for at least 3 months, at least 6 months, at least 9 months, at least 12 months, or at least 18 months.

The anti-IgE antibody as described herein “retains its biological activity” in an aqueous pharmaceutical formulation, if the biological activity of the antibody at a given time is within about 10% of the biological activity exhibited at the time the pharmaceutical formulation was prepared as determined in a potency assay, for example by determining the inhibition of IgE receptor binding, using methods known in the art.

In one embodiment, the anti-IgE antibody is stable in an aqueous pharmaceutical composition, wherein the biological activity of the anti-IgE antibody is between about 65% and 135% compared to a reference sample and wherein biological activity is assessed after storage at about 2-8° C. for at least 3 months, at least 6 months, at least 9 months, at least 12 months, or at least 18 months.

As used herein, an “aqueous” pharmaceutical composition is a composition suitable for pharmaceutical use, wherein the aqueous carrier is distilled water. A composition suitable for pharmaceutical use may be sterile, homogeneous and/or isotonic. Aqueous pharmaceutical compositions may be prepared either directly in an aqueous form, for example in pre-filled syringe ready for use or in a syringe prepared from a vial the comprises a pharmaceutical composition of the invention (the “liquid formulations”) or as lyophilisate to be reconstituted shortly before use. As used herein, the term “aqueous pharmaceutical composition” refers to the liquid formulation or reconstituted lyophilized formulation. In certain embodiments, the aqueous pharmaceutical compositions of the invention are suitable for ophthalmic administration to a human subject. In a specific embodiment, the aqueous pharmaceutical compositions of the invention are suitable for intravitreal administration.

The aqueous pharmaceutical compositions of the invention comprises, in addition to the anti-IgE antibody, further components such as one or more of the following: (i) a stabilizer; (ii) a buffering agent; (iii) a surfactant; and (iv) a pH of the composition of about 4.7 to about 5.2. Inclusion of each of such additional components can give compositions with low aggregation of the anti-IgE antibody. Preferably, the aqueous pharmaceutical compositions of the invention include, in addition to the anti-IgE antibody: (i) a stabilizer; (ii) a buffering agent; and (iii) a surfactant.

Suitable stabilizer for use with the invention can act, for example, as viscosity enhancing agents, bulking agents, solubilizing agents, and/or the like. The stabilizer can be ionic or non-ionic (e.g. sugars). As sugars they include, but are not limited to, monosaccharides, e.g., fructose, maltose, galactose, glucose, D-mannose, sorbose and the like; disaccharides, e.g. lactose, sucrose, trehalose, cellobiose, and the like; polysaccharides, e.g. raffinose, melezitose, maltodextrins, dextrans, starches, and the like; and alditols, such as mannitol, xylitol, maltitol, lactitol, xylitol sorbitol (glucitol) and the like. For example, the sugar may be sucrose, trehalose, raffinose, maltose, sorbitol or mannitol. The sugar may be a sugar alcohol or an amino sugar, such as sucrose or trehalose. Sucrose is preferred. As ionic stabilizer they may include salts such as NaCl. In a preferred embodiment, a sugar is present in the aqueous pharmaceutical composition of the invention, at a concentration of between 3 and 11% (w/v). In other embodiments, the sugar is trehalose at a concentration of about 5% to about 10%. In a preferred embodiment, the aqueous pharmaceutical composition comprises a concentration of about 7% to about 9% (w/v) trehalose. In another preferred embodiment, the aqueous pharmaceutical composition comprises a concentration of 8.5% (w/v) trehalose.

Suitable buffering agents for use with the invention include, but are not limited to, organic acid salts such as salts of citric acid, ascorbic acid, gluconic acid, carbonic acid, tartaric acid, succinic acid, acetic acid or phthalic acid; Tris, tromethamine hydrochloride, or phosphate buffer. In addition, amino acid components can also be used as buffering agent. Citrate or histidine buffer are particularly useful, including 10-30 mM of histidine buffer. In a preferred embodiment, the aqueous pharmaceutical composition comprises a buffering agent at a concentration of between about 1 and 60 mM, e.g., about 10-40 mM, about 15-30 mM, about 15-25 mM, about 10-20 mM, about 20 mM. In a preferred embodiment, the aqueous pharmaceutical composition comprises a buffering agent at a concentration of between about 1 and 60 mM, e.g., about 10-40 mM, about 15-30 mM, about 15-25 mM, about 10-20 mM, about 20 mM, wherein the buffering agent is a carboxylic acid buffer with pKa from about 4 to about 6. An example of carboxylic acid buffer with pKa from about 4 to about 6 is histidine, having pKa of 6.0. In yet another preferred embodiment, acetate with pKa of about 4.86) is another buffering agent. In certain embodiments, the buffering agent is histidine. In a preferred embodiment, the aqueous pharmaceutical composition comprises about 10-30 mM histidine, for example about 20 mM histidine.

In a preferred embodiment, the aqueous pharmaceutical composition comprises about 10-30 mM acetate, for example about 20 mM acetate.

The aqueous pharmaceutical compositions include such buffering agent or pH adjusting agent to provide improved pH control. In certain embodiment, an aqueous pharmaceutical composition of the invention has a pH between 4.5 to about 5.5. In one embodiment, the pH of an aqueous pharmaceutical composition of the invention is about 4.7 to about 5.2. In one embodiment, an aqueous pharmaceutical composition of the invention has a pH of about 4.5, about 4.7, about 4.8, about 4.9, about 5.0, about 5.1 about 5.2, about 5.3, about 5.4 or about 5.5. In a preferred embodiment, the aqueous pharmaceutical composition has a pH of about 4.7. In another preferred embodiment, the aqueous pharmaceutical composition has a pH of about 5.0. In another preferred embodiment, the aqueous pharmaceutical composition has a pH of about 5.2.

As used herein, the term “surfactant” herein refers to organic substances having amphipathic structures. Surfactants can be classified, depending on the charge of the surface-active moiety, into nonionic, anionic, cationic and dispersing agents for various pharmaceutical compositions and preparations of biological materials.

Suitable surfactants for use with the invention include, but are not limited to, non-ionic surfactants, ionic surfactants and zwitterionic surfactants. Typical surfactants for use with the invention include, but are not limited to, sorbitan fatty acid esters (e.g., sorbitan monocaprylate, sorbitan monolaurate, sorbitan monopalmitate), sorbitan trioleate, glycerine fatty acid esters (e.g., glycerine monocaprylate, glycerine monomyristate, glycerine monostearate), polyglycerine fatty acid esters (e.g., decaglyceryl monostearate, decaglyceryl distearate, decaglyceryl monolinoleate), polyoxyethylene sorbitan fatty acid esters (e.g.,. polyoxyethylene sorbitan monolaurate, polyoxyethylene sorbitan monooleate, polyoxyethylene sorbitan monostearate, polyoxyethylene sorbitan monopalmitate, polyoxyethylene sorbitan trioleate, polyoxyethylene sorbitan tristearate), polyoxyethylene sorbitol fatty acid esters (e.g., polyoxyethylene sorbitol tetrastearate, polyoxyethylene sorbitol tetraoleate), polyoxyethylene glycerine fatty acid esters (e.g., polyoxyethylene glyceryl monostearate), polyethylene glycol fatty acid esters (e.g., polyethylene glycol distearate), polyoxyethylene alkyl ethers (e.g., polyoxyethylene lauryl ether), polyoxyethylene polyoxypropylene alkyl ethers (e.g., polyoxyethylene polyoxypropylene glycol, polyoxyethylene polyoxypropylene propyl ether, polyoxyethylene polyoxypropylene cetyl ether), polyoxyethylene alkylphenyl ethers (e.g., polyoxyethylene nonylphenyl ether), polyoxyethylene hydrogenated castor oils (e.g., polyoxyethylene castor oil, polyoxyethylene hydrogenated castor oil), polyoxyethylene beeswax derivatives (e.g., polyoxyethylene sorbitol beeswax), polyoxyethylene lanolin derivatives (e.g., polyoxyethylene lanolin), and polyoxyethylene fatty acid amides (e.g., polyoxyethylene stearic acid amide); C₁₀-C₁₈ alkyl sulfates (e.g. sodium cetyl sulfate, sodium lauryl sulfate, sodium oleyl sulfate), polyoxyethylene C₁₀-C₁₈ alkyl ether sulfate with an average of 2 to 4 moles of ethylene oxide units added (e.g., sodium polyoxyethylene lauryl sulfate), and C₁-C₁₈ alkyl sulfosuccinate ester salts (e.g., sodium lauryl sulfosuccinate ester); and natural surfactants such as lecithin, glycerophospholipid, sphingophospholipids (e.g., sphingomyelin), and sucrose esters of C₁₂-₁₈ fatty acids. A composition may include one or more of these surfactants. Preferred surfactants are polyoxyethylene sorbitan fatty acid esters e.g. polysorbate 20, 40, 60 or 80. Polysorbate 80 or polyosorbate 20 is particularly preferred. In one embodiment, the aqueous pharmaceutical composition comprises 0.01% to 0.05% polysorbate 80, or polysorbate 20 (w/v). In another embodiment, the aqueous pharmaceutical composition comprises 0.01% to 0.03% polysorbate 80, or polysorbate 20 (w/v). In a preferred embodiment, the aqueous pharmaceutical composition comprises 0.01%, 0.02% or 0.03% polysorbate 20 (w/v). In one embodiment, the aqueous pharmaceutical composition comprises 0.01% polysorbate 80 (w/v). In one embodiment, the aqueous pharmaceutical composition comprises 0.02% polysorbate 80 (w/v). In another preferred embodiment, the aqueous pharmaceutical composition comprises 0.01% to 0.05% polysorbate 20 (w/v). In one embodiment, the aqueous pharmaceutical composition comprises 0.01% polysorbate 20 (w/v). In one embodiment, the aqueous pharmaceutical composition comprises 0.02% polysorbate 20 (w/v). In one embodiment, the aqueous pharmaceutical composition comprises 0.03% polysorbate 20 (w/v).

Other contemplated excipients, which may be utilized in the aqueous pharmaceutical compositions of the invention include, for example, antimicrobial agents, antioxidants, antistatic agents, lipids such as phospholipids or fatty acids, steroids such as cholesterol, protein excipients such as serum albumin (human serum albumin), recombinant human albumin, gelatin, casein, salt-forming counterions such sodium and the like. These and additional known pharmaceutical excipients and/or additives suitable for use in the formulations of the invention are known in the art, e.g., as listed in “The Handbook of Pharmaceutical Excipients, 4th edition, Rowe et al., Eds., American Pharmaceuticals Association (2003); and Remington: the Science and Practice of Pharmacy, 21^{st} edition, Gennaro, Ed., Lippincott Williams & Wilkins (2005).

In certain embodiments, lyophilisation of an anti-IgE antibody is contemplated to provide an aqueous pharmaceutical composition of the invention for treating a subject in need thereof.

Techniques for lyophilisation of antibodies are well known in the art e.g. see John F. Carpenter and Michael J. Pikal, 1997 (Pharm. Res. 14, 969-975); Xialin (Charlie) Tang and Michael J. Pikal, 2004 (Pharm. Res. 21, 191-200). Accordingly, in one embodiment provided is a lyophilized formulation prepared by lyophilizing the aqueous pharmaceutical composition described herein. In another embodiment, provided is a method for preparing a lyophilisate, comprising the steps of: (i) preparing an aqueous pharmaceutical composition comprising an anti-VEGF antibody as described herein and (ii) lyophilizing the aqueous solution.

Before a lyophilisate can be administered to a patient it should be reconstituted with an aqueous reconstituent. This step permits antibody and other components in the lyophilisate to re-dissolve to give a solution which is suitable for injection to a patient.

The volume of aqueous material used for reconstitution dictates the concentration of the antibody in a resulting pharmaceutical composition. Reconstitution with a smaller volume of reconstituent than the pre-lyophilisation volume provides a composition which is more concentrated than before lyophilisation. The reconstitution factor (volume of formulation after lyophilization:volume of formulation before lyophilization) may be from 1:0.5 to 1:6. A reconstitution factor of 1:3 is useful. As mentioned above, lyophilisates of the invention can be reconstituted to give aqueous compositions with an anti-IgE antibody concentration of at least 50 mg/ml (i.e., at least 60, 72, 80, 90, 100, 110, 120, or 130 mg/ml), and the volume of reconstituent will be selected accordingly. If required, the reconstituted formulation can be diluted prior to administration to a patient as appropriate to deliver the intended dose.

Typical reconstituents for lyophilized antibodies include sterile water or buffer, optionally containing a preservative. If the lyophilisate includes a buffering agent then the reconstituent may include further buffering agent (which may be the same as or different from the lyophilisate's buffering agent) or it may instead include no buffering agent (e.g. WFI (water for injection), or physiological saline).

The aqueous pharmaceutical composition described herein may be in the form of a liquid. In a preferred embodiment, the aqueous pharmaceutical composition is in the form of a liquid. In one embodiment, the aqueous pharmaceutical composition is comprised as a liquid in a vial.

The aqueous pharmaceutical compositions of the invention comprising anti-IgE antibodies can be used to treat a variety of diseases or disorders. Pharmaceutical compositions comprising anti-IgE antibodies are particularly useful to treat allergies, food allergy.

The terms “treat,” “treating,” and “treatment,” as used herein refer to therapeutic measures described herein. The methods of “treatment” employ administration to a subject, in need of such treatment, an antibody of the present invention, for example, a subject having a IgE-mediated disorder or a subject who ultimately may acquire such a disorder, in order to prevent, cure, delay, reduce the severity of, or ameliorate one or more symptoms of the disorder or recurring disorder, or in order to prolong the survival of a subject beyond that expected in the absence of such treatment.

Aqueous pharmaceutical compositions of the invention can be administered to a patient. As used herein, the term “subject” or “patient” refers to human and non-human mammals, including but, not limited to, primates, rabbits, pigs, horses, dogs, cats, sheep, and cows. Preferably, a subject or patient is a human.

Administration will typically be via a syringe. Thus the invention provides a delivery device (e.g., a syringe) including a pharmaceutical composition of the invention (e.g., pre-filled syringe), and a kit comprising a syringe and a vial that includes a pharmaceutical composition of the invention. Patients will receive an effective amount of the anti-IgE antibody as the principal active ingredient (i.e., an amount that is sufficient to achieve or at least partially achieve the desired effect). A therapeutically effective dose is sufficient if it can produce even an incremental change in the symptoms or conditions associated with the disease. The therapeutically effective dose does not have to completely cure the disease or completely eliminate symptoms. Preferably, the therapeutically effective dose can at least partially arrest the disease and its complications in a patient already suffering from the disease. Amounts effective for this use will depend upon the severity of the disorder being treated and the general state of the patient's own immune system.

The dose amount can be readily determined using known dosage adjustment techniques by a physician having ordinary skill in treatment of the disease or condition. The therapeutically effective amount of an anti-IgE antibody used in an aqueous pharmaceutical composition of the invention is determined by taking into account the desired dose volumes and mode(s) of administration, for example. Typically, therapeutically effective compositions are administered in a dosage ranging from 0.001 mg/ml to about 200 mg/ml per dose. Preferably, a dosage used in a method of the invention is about 60 mg/ml to about 120 mg/ml (i.e., about 60, 72, 80, 90, 100, 110, or 120 mg/ml). In a preferred embodiment, the dosage of an anti-IgE antibody used in a method of the invention is 72 mg/0.6ml or 120 mg/ml.

The invention also provides formulations (i.e., aqueous pharmaceutical compositions) of the invention for use as medicaments, e.g., for use in delivering an antibody to a patient, or for use in treating or ameliorating one or more of the diseases and disorders described above.

The invention further provides a method for delivering an anti-IgE antibody to a patient, comprising a step of administering to the patient an aqueous pharmaceutical composition of the invention.

In certain embodiments, a method for delivering an anti-IgE antibody to a patient invention comprises the steps of: (i) reconstituting a lyophilisate of the invention to give an aqueous formulation, and (ii) administering the aqueous formulation to the patient. Step (ii) ideally takes place within 24 hours of step (i) (e.g., within 12 hours, within 6 hours, within 3 hours, or within 1 hour).

In one embodiment, the aqueous pharmaceutical composition is comprised in a vial. In another embodiment, the aqueous pharmaceutical composition is comprised in a delivery device. In one embodiment, such delivery device is a pre-filled syringe. In one embodiment a method for delivering an anti-IgE antibody to a patient comprises administering the aqueous pharmaceutical composition by s.c. injection.

As used herein, the term “an automated disposable injection device”, or an “auto-injector” refers to a device for pre-filled glass or polymer syringes suitable for delivery of liquid drugs, such antibody formulations, to all patient groups. Exemplary auto-injector are YpsoMate auto-injectors.

Certain specific embodiments of the invention are described as numbered hereafter:
1. A stable aqueous pharmaceutical composition comprising:
   at least 50 mg/ml to about 150 mg/ml of an anti-IgE antibody, such as ligelizumab,
   about 4.5% to 11% (w/v) trehalose,
   about 5-25 mM carboxylic acid buffer with pKa from about 4 to about 6, preferably histidine or acetate, and
   from about 0.01% to about 0.05% polysorbate 20 (w/v) or from about 0.01% to about 0.05% polysorbate 80 (w/v),
   wherein the pH of the composition is about 4.7 to about 5.2.
2. The aqueous pharmaceutical composition according to embodiment 1, wherein the anti-IgE antibody is ligelizumab.
3. The aqueous pharmaceutical composition according to embodiment 1, wherein the anti-IgE antibody is an antibody which has demonstrated to be biosimilar to or interchangeable to ligelizumab.
4. The aqueous pharmaceutical composition of any of the preceding embodiments, wherein the viscosity of the composition is from about 5 to about 30 mPa-s, preferably not more that about 20 mPa-s (from about 5 to about 20 mPa-s).
5. The aqueous pharmaceutical composition of any of the preceding embodiments, wherein the pH of the composition is about 4.8.
6. The aqueous pharmaceutical composition of any of the preceding embodiments, wherein the pH of the composition is about 4.9.
7. The aqueous pharmaceutical composition of any of the preceding embodiments, wherein the pH of the composition is about 5.0.
8. The aqueous pharmaceutical composition of any of the preceding embodiments, wherein the pH of the composition is about 5.1.
9 The aqueous pharmaceutical composition of any of the preceding embodiments, wherein the pH of the composition is about 5.2.
10. The aqueous pharmaceutical composition of any of the preceding embodiments, comprising between about 60 mg/ml and about 120 mg/ml of the anti-IgE antibody.
11. The aqueous pharmaceutical composition of embodiment 10, comprising about 120 mg/ml of the anti-IgE antibody.
12. The aqueous pharmaceutical composition of any of the preceding embodiments, comprising about 0.01%-0.03% polysorbate 20 (w/v).
13. The aqueous pharmaceutical composition of embodiment 12, comprising about 0.02% polysorbate 20 (w/v).
14. The aqueous pharmaceutical composition of any of the preceding embodiments, comprising about 10-25 mM histidine buffer.
15. The aqueous pharmaceutical composition of embodiment 14, comprising about 20 mM histidine buffer.
16. The aqueous pharmaceutical composition of any of the preceding embodiments, comprising about 5.5%-9.0% (w/v) trehalose.
17. The aqueous pharmaceutical composition of embodiment 16, comprising about 8.5% (w/v) trehalose.
18. The aqueous pharmaceutical composition of any of the preceding embodiments, comprising 8.5% (w/v) trehalose, 20 mM histidine, 0.02% (w/v) polysorbate 20, and wherein the pH of the composition is about 5.0.
19. The aqueous pharmaceutical composition of any of the embodiments 1 to 15, comprising about 200 -270 mM trehalose.
20. The aqueous pharmaceutical composition of any of the embodiments 1 to 15, comprising about 250 -270 mM trehalose, preferably about 250 mM trehalose.
21. A stable aqueous pharmaceutical composition comprising about 120 mg/ml ligelizumab, about 250 mM trehalose, about 20 mM histidine, about 0.02% (w/v) polysorbate 20, and wherein the pH of the composition is about 5.0.
22. The aqueous pharmaceutical composition of any of the preceding embodiments, wherein said composition is stable for at least 18 months at 2-8° C.
23. The aqueous pharmaceutical composition of any of the preceding embodiments, wherein said composition is liquid.
24 The aqueous pharmaceutical composition of any of the preceding embodiments, wherein said composition does not contain arginine, preferably arginine-HC1.
25. The aqueous pharmaceutical composition of any of the preceding embodiments, wherein said composition does not contain arginine-HCl in an amount of 50-200 mM.
26. A method for delivering an anti-IgE antibody to a subject, comprising administering to said subject the aqueous pharmaceutical composition of any of embodiments 1-25.
27. A method of treating an allergy that is mediated by IgE, comprising administering to a subject the aqueous pharmaceutical composition of any of embodiments 1-25.
28. The method of embodiment 27, wherein said allergy is food allergy.
29. A method of treating, preventing, or reducing anaphylaxis, e.g., IgE mediated anaphylaxis, comprising administering to a subject in need thereof the aqueous pharmaceutical composition of any of embodiments 1-25.
30. A method of treating, preventing or reducing IgE-mediated allergic reaction in a subject with recurrent spontaneous anaphylaxis due to unknown and/or unavoidable triggers, comprising administering to a subject in need thereof the aqueous pharmaceutical composition of any of embodiments 1-25.
31. A method of treating, preventing or reducing severe or serious allergic IgE-mediated reactions triggered by at least one allergen (e.g. insect stings/bites, venom, medications, e.g. beta-lactam antibiotics, NSAIDS, biological agents; aeroallergens, occupational allergens, radiocontrast media, natural rubber latex, seminal fluid), comprising administering to a subject in need thereof the aqueous pharmaceutical composition of any of embodiments 1-25.
32. A method of treating, preventing or reducing severe or serious allergic IgE-mediated reactions of unidentified triggers (idiopathic anaphylaxis) comprising administering to a subject in need thereof the aqueous pharmaceutical composition of any of embodiments 1-25.
33. The method of any of embodiments 26-32, wherein said administration is s.c.
34. An aqueous pharmaceutical composition of any one of embodiments 1-25 for use in delivering an anti-IgE antibody to a subject, comprising a step of administering the aqueous pharmaceutical composition to the subject.
35. An aqueous pharmaceutical composition of any one of embodiments 1-25 for use in treating an allergy that is mediated by IgE, comprising administering the aqueous pharmaceutical composition to a subject.
36. An aqueous pharmaceutical composition for use according to embodiment 35, wherein said allergy is food allergy.
37. An aqueous pharmaceutical composition for use according to any of embodiments 34-36, wherein said administration is s.c, preferably using an auto-injector.
38. A dosage form comprising the aqueous pharmaceutical composition of any of the embodiments 1-25.
39. A delivery device comprising the aqueous pharmaceutical composition of any of embodiments 1-25.
40. The delivery device of embodiment 39, which is a pre-filled syringe.
41. The delivery device of embodiment 39, which is an automated disposable injection device, such as an auto-injector.

The skilled person realizes that the features, aspects and embodiments taught in the text are all combinable with each other and particular aspects combining features and/or embodiments from various parts of the text will be considered to be adequately disclosed to the skilled person.

It is to be understood that each embodiment may be combined with one or more other embodiments, to the extent that such a combination is consistent with the description of the embodiments. It is further to be understood that the embodiments provided above are understood to include all embodiments, including such embodiments as result from combinations of embodiments.

As used herein, all percentages are percentages by weight, unless stated otherwise.

As used herein and unless otherwise indicated, the terms “a” and “an” are taken to mean “one”, “at least one” or “one or more”. Unless otherwise required by context, singular terms used herein shall include pluralities and plural terms shall include the singular.

As used herein, the term “comprising” encompasses “including” as well as “consisting” and “essentially consisting of”, e.g., a composition comprising X may consist exclusively of X or may include something additional, e.g., X+Y.

The term “or” is used herein to mean, and is used interchangeably with the term “and/or”, unless context clearly indicates otherwise.

The contents of any patents, patent applications, and references cited throughout this specification are hereby incorporated by reference in their entireties.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

### EXAMPLES

The following examples describe formulation development efforts designed to identify suitable stabilization approaches and compositions to provide stable, highly concentrated solutions comprising the antibody ligelizumab, enabling a formulation with at least an 12-month shelf-life at refrigerated storage conditions that meets the regulatory requirements for pharmaceutical products.

The following examples summarize the formulation development of 72 and 120 mg/ml ligelizumab solutions stable at 2-8° C. storage for at least 18 months. The formulation development effort focused on inhibition of the formation of aggregation particles and meeting the USP requirement for content, purity and potency.

### ANALYTICAL METHODS

The following methods were used throughout the Examples as indicated: SEC Size Exclusion Chromatography method or SE-HPLC (Size exclusion chromatography), CEX Cation Exchange Chromatography, Bioanalyzer (sum of degradation products), RP-HPLC, Turbidity (NTU), viscosity [mPa·s], Molecular weight by laser light scattering, Particle measurement (by Light obscuration (LO)), Osmolality [mOsm/kg], Purity by rCE-SDS, Potency: Inhibition of IgE receptor binding, CEX.

### Example 1. Formulation Screening Studies

A liquid formulation was developed as liquid formulation for ligelizumab, suitable for s.c. injection as preferred route of administration.

### pH Refinement Study

The pH refinement study focused on the evaluation of pH-range 5.0 to 5.5 at a ligelizumab concentration of 150 mg/mL to evaluate the optimal pH value at this protein concentration and to check for the possibility of a slightly increased pH leading to improved buffering capacity of the histidine buffer.

The pH, the appearance by visual observation, and the sum of degradation products were used as evaluation criteria after storing the tested formulation at 40° C. for 4 weeks. Precipitation occurred at pH 5.50. Therefore, for the initial screen 2 a pH ≤5.25 was selected. A set of possible 12 liquid formulations was tested.

TABLE 1
Details on the formulations tested

| | Ligelizumab | Histidine | | | Stabilizer | | Surfactant |
|---|---|---|---|---|---|---|---|
| | concentration | concentration | | | concentration | | concentration |
| Formulation | [mg/mL] | [mM] | pH | Stabilizer | [mM] | Surfactant | [% (w/v)] |
| 1 | 115 | 20 | 5.0 | Mannitol | 300 | PS80 | 0.04 |
| 2 | 115 | 20 | 5.0 | Mannitol | 300 | PS20 | 0.04 |
| 3 | 115 | 20 | 5.0 | Mannitol | 300 | Pol188 | 0.3 |
| 4 | 115 | 20 | 5.0 | Trehalose | 300 | PS80 | 0.04 |
| 5 | 115 | 20 | 5.0 | Glycine | 300 | PS80 | 0.04 |
| 6 | 115 | 20 | 5.0 | Arginine | 150 | PS80 | 0.04 |
| 7 | 115 | 20 | 5.0 | Arginine | 150 | PS20 | 0.04 |
| 8 | 115 | 20 | 5.0 | Arginine | 150 | Pol188 | 0.3 |
| 9 | 115 | 20 | 5.0 | NaCl | 150 | PS80 | 0.04 |
| 10 | 115 | 20 | 5.0 | NaCl | 150 | PS20 | 0.04 |
| 11 | 115 | 20 | 5.0 | NaCl | 150 | Pol188 | 0.3 |
| 12 | 115 | 20 | 5.0 | Na₂SO₄ | 100 | PS80 | 0.04 |

TABLE 2
SEC Main Peak

| Condition | | | |
|---|---|---|---|
| T0 | 5° C. | 25° C. | 40° C. |

| Pull point [M] | 0 | 3 | 3 | 1.5 | 3 |
|---|---|---|---|---|---|
| 1 | 99.13 | 99.38 | 99.24 | 96.96 | 96.18 |
| 2 | 99.14 | 99.44 | 99.30 | 97.20 | 96.55 |
| 3 | 99.20 | 99.40 | 99.25 | 97.10 | 96.25 |
| 4 | 99.12 | 99.45 | 99.22 | 97.10 | 96.36 |
| 5 | 99.20 | 99.43 | 99.14 | 97.13 | 95.88 |
| 6 | 99.23 | 99.42 | 99.49 | 97.39 | 96.12 |
| 7 | 99.22 | 99.45 | 99.44 | 97.51 | 96.28 |
| 8 | 99.21 | 99.36 | 99.44 | 97.35 | 96.23 |
| 9 | 99.18 | 99.40 | 99.23 | 96.64 | 95.69 |
| 10 | 99.20 | 99.46 | 99.28 | 96.66 | 95.78 |
| 11 | 99.14 | 99.40 | 99.12 | 96.62 | 95.75 |
| 12 | 99.20 | 99.35 | 99.21 | 96.63 | 95.30 |

All numbers are given as [%]

TABLE 3
SEC Aggregation products

| Condition | | | |
|---|---|---|---|
| T0 | 5° C. | 25° C. | 40° C. |

| Pull point [M] | 0 | 3 | 3 | 1.5 | 3 |
|---|---|---|---|---|---|
| 1 | 0.87 | 0.62 | 0.76 | 1.27 | 1.13 |
| 2 | 0.86 | 0.56 | 0.70 | 1.15 | 1.05 |
| 3 | 0.80 | 0.60 | 0.75 | 1.38 | 1.17 |
| 4 | 0.88 | 0.55 | 0.78 | 1.26 | 1.08 |
| 5 | 0.80 | 0.57 | 0.86 | 1.40 | 1.54 |
| 6 | 0.77 | 0.58 | 0.51 | 0.82 | 0.82 |
| 7 | 0.78 | 0.55 | 0.56 | 0.73 | 0.62 |
| 8 | 0.79 | 0.64 | 0.56 | 0.92 | 0.80 |
| 9 | 0.82 | 0.60 | 0.77 | 1.27 | 1.36 |
| 10 | 0.80 | 0.54 | 0.72 | 1.42 | 1.35 |
| 11 | 0.86 | 0.60 | 0.88 | 1.51 | 1.33 |
| 12 | 0.80 | 0.65 | 0.79 | 1.36 | 1.35 |

All numbers are given as [%]

TABLE 4
SEC Degradation products

| Condition | | | |
|---|---|---|---|
| T0 | 5° C. | 25° C. | 40° C. |

| Pull point [M] | 0 | 3 | 3 | 1.5 | 3 |
|---|---|---|---|---|---|
| 1 | n.d. | n.d. | 0.00 | 1.77 | 2.69 |
| 2 | n.d. | n.d. | 0.00 | 1.65 | 2.40 |
| 3 | n.d. | n.d. | 0.00 | 1.52 | 2.58 |
| 4 | n.d. | n.d. | 0.00 | 1.64 | 2.57 |
| 5 | n.d. | n.d. | 0.00 | 1.47 | 2.57 |
| 6 | n.d. | n.d. | 0.00 | 1.79 | 3.06 |
| 7 | n.d. | n.d. | 0.00 | 1.76 | 3.10 |
| 8 | n.d. | n.d. | 0.00 | 1.72 | 2.96 |
| 9 | n.d. | n.d. | 0.00 | 2.09 | 2.95 |
| 10 | n.d. | n.d. | 0.00 | 1.92 | 2.86 |
| 11 | n.d. | n.d. | 0.00 | 1.87 | 2.92 |
| 12 | n.d. | n.d. | 0.00 | 2.02 | 3.35 |

All numbers are given as [%]
n.d. not detectable

TABLE 5
RP-HPLC Main Peak

| Condition | | | |
|---|---|---|---|
| T0 | 5° C. | 25° C. | 40° C. |

| Pull point [M] | 0 | 3 | 3 | 1.5 | 3 |
|---|---|---|---|---|---|
| 1 | 95.51 | 95.88 | 94.79 | 89.89 | 85.87 |
| 2 | 95.32 | 96.15 | 94.80 | 89.97 | 85.60 |
| 3 | 95.52 | 95.73 | 94.84 | 90.44 | 86.17 |
| 4 | 95.50 | 95.68 | 94.69 | 90.11 | 86.21 |
| 5 | 95.75 | 95.71 | 94.28 | 90.40 | 86.34 |
| 6 | 95.49 | 95.87 | 94.67 | 91.12 | 86.12 |
| 7 | 95.35 | 95.60 | 94.80 | 90.71 | 87.10 |
| 8 | 95.59 | 95.50 | 94.74 | 91.17 | 86.48 |
| 9 | 95.65 | 95.52 | 95.20 | 90.79 | 85.69 |
| 10 | 95.59 | 95.06 | 95.05 | 90.16 | 85.61 |
| 11 | 95.66 | 95.03 | 94.91 | 90.41 | 86.86 |
| 12 | 95.39 | 94.81 | 94.80 | 90.37 | 84.63 |

All numbers are given as [%]

The impact of the screened stabilizers (mannitol, trehalose, glycine), salts (arginine hydrochloride, sodium chloride, sodium sulfate), and surfactants (polysorbate 20 and 80, poloxamer 188) on the tested quality attributes of the formulations was analyzed. A less favorable stability behavior of formulations 6, 7 and 8, was observed at 40° C. storage at SEC aggregation products and SEC degradation products (data not shown).

A follow-up study with higher sensitivity was designed. Formulations with a higher pH value (5.5, 5.75, and 6.0) were also included, to evaluate the solubility at these pH-values in the presence of the selected stabilizers. To allow on the one hand selection of the best formulation variants within a short time frame (3 m) and on the other hand collecting of stability data over a longer time period, a thorough stability program up to 6m (plus an additional back-up at 5° C.) was set up.

TABLE 6
Details on the formulations tested in the focus screen

| | | | | Polysorbate | Poloxamer | | |
|---|---|---|---|---|---|---|---|
| | Ligelizumab | Mannitol | Trehalose | 20 | 188 | Histidine | pH- |
| Formulation | [mg/mL] | [mM] | [mM] | [% (w/v)] | [% (w/v)] | [mM] | value |
| 9834.01.DN | 150.0 | 200 | — | 0.02 | — | 10 | 5.25 |
| 9834.02.DN | 150.0 | 200 | — | 0.06 | — | 30 | 5.25 |
| 9834.03.DN | 150.0 | 350 | — | — | 0.1 | 10 | 5.25 |
| 9834.04.DN | 150.0 | 350 | — | — | 0.3 | 30 | 5.25 |
| 9834.05.DN | 150.0 | 270 | — | 0.04 | — | 20 | 5.25 |
| 9834.06.DN | 150.0 | 270 | — | — | 0.2 | 20 | 5.25 |
| 9834.07.DN | 150.0 | — | 350 | 0.06 | — | 10 | 5.25 |
| 9834.08.DN | 150.0 | — | 200 | 0.02 | — | 30 | 5.25 |
| 9834.09.DN | 150.0 | — | 200 | — | 0.3 | 10 | 5.25 |
| 9834.10.DN | 150.0 | — | 350 | — | 0.1 | 30 | 5.25 |
| 9834.11.DN | 150.0 | — | 270 | 0.04 | — | 20 | 5.25 |
| 9834.12.DN | 150.0 | — | 270 | — | 0.2 | 20 | 5.25 |
| 9834.13.DN | 150.0 | 270 | — | 0.04 | — | 20 | 5.50 |
| 9834.14.DN | 150.0 | 270 | — | 0.04 | — | 20 | 5.75 |
| 9834.15.DN | 150.0 | 270 | — | 0.04 | — | 20 | 6.00 |

During adjusting the pH of formulation 15 to pH 6.00 precipitation occurred. Therefore, this formulation was cancelled from the stability plan. Subsequently also formulation 14 precipitated during storage at 5° C. The same happened to formulation 13 during shaking at 150 rpm at RT and during storage at 5° C. All these formulations were subsequently cancelled from the stability plan.

TABLE 7
Viscosity

| | Condition |
|---|---|
| | T0 |
| Pull point [M] | 0 |
| 9834.01.DN | 17.8 |
| 9834.02.DN | 21.3 |
| 9834.03.DN | 36.8 |
| 9834.04.DN | 24.1 |
| 9834.05.DN | 25.5 |
| 9834.06.DN | 25.4 |
| 9834.07.DN | 34.4 |
| 9834.08.DN | 24.2 |
| 9834.09.DN | 36.1 |
| 9834.10.DN | 28.7 |
| 9834.11.DN | 29.4 |
| 9834.12.DN | 28.7 |
| 9834.13.DN | n.m. ¹ |
| 9834.14.DN | — |
| 9834.15.DN | — |

All numbers are given as [mPa · s]
—: not tested
¹ n.m.: not measured, as the sample was gel

Increase of molecular wight average after freeze/thaw stress (5 cycles from −20° C. to room temperature (RT)) was detected for formulations 9834.01.DN, 9834.02.DN, 9834.03.DN, 9834.04.DN, 9834.05.DN, 9834.06.DN, and 9834.09.DN. This indicates formation of aggregates of the protein for these formulations. Overall, the results of formulations 9834.06.DN, 9834.07.DN, 9834.08.DN, 9834.10.DN, 9834.11.DN, 9834.12.DN indicate a better suitability for trehalose as stabilizer compared to mannitol.

Least amount of particles after shake stress (shaking @ 150 rpm: 1 week at RT) was observed for formulations 9834.07.DN and 9834.08.DN.

Regarding the concentration of trehalose, measurements at 6 M pull point (25° C.) were performed for formulations 9834.07.DN, 9834.08.DN and 9834.11.DN. For formulations 9834.11.DN, containing 270 mM of trehalose, less particles were measured than for formulations 9834.07.DN and 9834.08.DN, containing higher or lower concentrations of trehalose after 6 M of storage at 25° C.

The selection of the formulation to be further evaluated in the optimization screen was based on the lead candidate formulation which was selected at the end of focused screen (150 mg/ml ligelizumab, 270 mM Trehalose, 0.02% polysorbate 20, and 10 mM histidine). However, since histidine is not optimal to buffer at pH 5.0 due to its pKa of 6.0, it was decided to include acetate (pKa: 4.86) in the optimization screen as a potential alternative buffering agent. Two different acetate concentrations were assessed, 10 and 20 mM, with the formulation containing 20mM acetate buffer tested at pH 4.7 and 5.3 in addition to pH 5.0 in order to assess the pH robustness of ligelizumab in a likely pH range at the end of potential shelf life specification.

TABLE 8
Formulation to be tested:

| Formulation No. | | Ligelizumab | Trehalose | Tween 20 | Histidine | Acetate | |
|---|---|---|---|---|---|---|---|
| Lab-code | | [mg/mL] | [mM] | [% (w/v)] | [mM] | [mM] | pH |
| 10110.01.SR | 1 (vial) | 150 | 270 | 0.02 | 10 | — | 5.0 |
| | 1 (syringe) | | | | | | |
| 10110.02.SR | 2 (vial) | 150 | 270 | 0.02 | 20 | — | 5.0 |
| | 2 (syringe) | | | | | | |
| 10110.03.SR | 3 (vial) | 150 | 270 | 0.02 | — | 10 | 5.0 |
| | 3 (syringe) | | | | | | |
| 10110.04.SR | 4 (vial) | 150 | 270 | 0.02 | — | 20 | 5.0 |
| | 4 (syringe) | | | | | | |
| 10110.05.SR | 5 (vial) | 150 | 270 | 0.02 | — | 20 | 4.7 |
| 10110.06.SR | 6 (vial) | 150 | 270 | 0.02 | — | 20 | 5.3 |

No relevant differences between formulations 1-4 could be detected during the stability program, except for the viscosity, turbidity and osmolality and RP-HPLC. The viscosity of the formulations containing histidine was lower than that of the formulations containing acetate. Furthermore, the viscosity was inversely proportional to the histidine concentration. Also, formulations 5 and 6, which were set at different pH (4.7 and 5.3, however, using acetate as buffer, instead of histidine) differed from formulations 1-4, when assessed for viscosity, turbidity and osmolality (results not shown. Based on these results, the formulation containing 20 mM histidine (formulation 10110.02.SR) was chosen for further screening. However, this formulation needed to be adapted to be compatible with manufacturing processes, therefore the formulation was adapted to: 140mg/mL ligelizumab, 20 mM histidine, 250 mM Trehalose. A further optimization screen was performed to investigate the impact of pH and concentration of ligelizumab and histidine on the viscosity of the formulation.

TABLE 9
Evaluation of viscosity

| | | Ligelizumab | | Trehalose | Polysorbate | Results: |
|---|---|---|---|---|---|---|
| Formulation | | concentration | Histidine | dihydrate | 20 | Viscosity |
| No. | pH | [mg/mL] | [mM] | [mM] | [%] | [mPa · s] ¹ |
| 12130.09 | 4.7 | 100 | 10 | 250 | 0.02 | 8.88 |
| 12130.12 | | 100 | 30 | | | 7.09 |
| 12130.04 | | 120 | 20 | | | 12.67 |
| 12130.03 | | 140 | 10 | | | 26.44 |
| 12130.14 | | 140 | 30 | | | 19.04 |
| 12130.13 | 5 | 100 | 20 | | | 7.31 |
| 12130.10 | | 120 | 10 | | | 13.81 |
| 12130.06 | | 120 | 20 | | | 12.97 |
| 12130.02 | | 120 | 30 | | | 10.92 |
| 12130.01 | | 140 | 20 | | | 23.31 |
| 12130.016 | 5.3 | 100 | 10 | | | 8.12 |
| 12130.08 | | 100 | 30 | | | 6.48 |
| 12130.015 | | 120 | 20 | | | 11.08 |
| 12130.05 | | 140 | 10 | | | 25.83 |
| 12130.07 | | 140 | 30 | | | 17.34 |

TABLE 10
Potency: Inhibition of IgE receptor binding of 2 batches of the of the formulation 12130.06

| | Condition | | | | | | | | | | | | | | | | | | | |
|---|---|---|---|---|---|---|---|---|---|---|---|---|---|---|---|---|---|---|---|---|
| | T0 | 5° C. | | | | | | | | | | | 25° C./60% RH | | | | | 40° C./75% RH | | |
| | Pull point [M] | | | | | | | | | | | | | | | | | | | |
| | 0 | 1.5 | 3 | 6 | 9 | 12 | 18 | 24 | 36 | 42 | 48 | 60 | 1.5 | 3 | 6 | 9 | 12 | 1.5 | 3 | 6 |
| Batch 1 | 110 | — | 102 | 105 | — | 108 | — | 104 | — | — | — | — | — | 108 | 101 | — | — | — | — | — |
| Batch 2 | 104 | 98 | 96 | 104 | 108 | 92 | 111 | 105 | 98 | 93 | 99 | 99 | 101 | 95 | 103 | 107 | 94 | 95 | 96 | 98 |

All numbers are given as [%] (relative biological activity compared to the reference substance)
Specifications: 70-130%
Specification limits are applicable only for long-term storage conditions
For t = 0, release values were taken
—: not tested

A statistical evaluation (using the software JMP, version 14.2.0.) of the results showed that all three evaluated factors (pH, API concentration, and histidine concentration) did have a significant impact on the viscosity. The viscosity is the higher, the lower the pH, the lower the histidine concentration, and the higher the ligelizumab concentration. The evaluation has showed that for 120 mg/mL ligelizumab, 20 mM histidine and pH 5.0 the predicted viscosity and the 95% confidence interval is well below 20 mPa·s. When fixing pH to 5.0 and histidine to 20 mM, ligelizumab concentration must not be more than 135.15 mg/mL in order to be 95% confident that the viscosity will not exceed 20 mPa·s. furthermore, when fixing pH to 4.7 (inducing maximal viscosity) and histidine to 10 mM (inducing maximal viscosity), ligelizumab concentration must not be more than 128 mg/mL in order to be 95% confident that the viscosity will not exceed 20 mPa·s.

Based on the results, as the viscosity of the ligelizumab solution needed to be lower than 20 mPa·s, so that it could be used in an autoinjector, formulation 12130.06, consisting of 120 mg/mL ligelizumab, 20 mM histidine, 250 mM trehalose and 0.02% Polysorbate 20 at pH 5.0, is preferred.

### Storage at 5° C. (Long-Term Storage Condition) and Storage at 25° C./60% Relative Humidity (RH) (Accelerated Storage Condition for Clinical Batches)

After 24 months storage at 5° C. all results are still within the requirements defined for long-term storage for clinical formulations. After 60 months storage at 5° C. all results are within the requirements defined for long-term storage.

After 6 months storage at 25° C./60% RH, except purity by SEC all results are still within the requirements defined for long-term storage.

After 12 months storage at 25° C./60% RH, as expected due to the accelerated storage some of the results are not within the requirements defined for long-term storage, due to the stress storage conditions.

Those results confirm the suitability of the selected formulation for ligelizumab 120 mg/mL to meet the regulatory requirements for pharmaceutical products. The histidine buffer was chosen as pH buffer, the selected concentration of 20 mM showed the preferred viscosity for the intended use of the ligelizumab formulation. Polysorbate 20 was chosen, as the formulations showed better stability behavior than those containing Poloxamer 188. Trehalose was selected as stabilizer: at a concentration of 270 mM it showed best results in the focus screen, which were confirmed during optimization screen 1. Due to the lowered final concentration of ligelizumab of 120 mg/mL (compared to 150 mg/mL in the focus screen and optimization screen 1), the concentration of trehalose was adapted to 250 mM.

### Example 2. Formulation Syringe Combinations Study

The 120 mg/mL ligelizumab formulation pH was buffered to 5. As described herein, the importance of the construction of syringe materials on the compatibility with sensitive antibody formulations is critical. In particular, the impact of lubricants or the absence thereof, on particles in the antibody formulation. Furthermore, a comparison was made on the force needed to break-loose and glide a plunger down three different syringe barrels.

A customized 0.2 mL transfer syringe with ten to hundred times lower dimensional variability compared to ordinary glass was used. An integrated luer-syringe barrel design reduced the dead volume in the luer tip. The small volume 0.2 mL barrel wasted far less drug compared to the traditional 1ml syringe format. These combined design enhancements permitted a drug injection volume of 0.02 mL within a 5% dose accuracy. Additionally, an innovative lubricant was developed to replace silicone oil on the syringe barrel with fewer oil-like particles leaching into the drug product. The lubricant was deposited by a unique plasma enhanced chemical vapor deposition process (PECVD).

### Syringes and Plungers

Three different 1 mL staked needle syringe combinations were used in this study. Two of those combinations were SiO2 hybrid syringes, referred to as SiO2 (PECVD) and SiO2 (NONE). SiO2 (PECVD) syringes were coated with a PECVD barrier coating system and a PECVD lubricant. A West Novapure® Plunger was used with the SiO2 (PECVD) syringes. SiO2 (NONE) syringes were lubricant free and only coated with a PECVD three-layer barrier coating system. A proprietary lubricant free plunger was used with the SiO2 (NONE) syringes. Both SiO2 (PECVD) and SiO2 (NONE) syringes were molded from cyclic olefin polymer (COP) with an embedded 27-gauge needle.

Benchmark borosilicate glass syringes, referred to as Glass (Silicone), were coated with spray-on silicone oil lubricant on the syringe barrels. West Novapure® plungers were used with the Glass (Silicone) syringes.

Drug stability was evaluated at accelerated conditions after 3 and 6 months at 25° C. and after 1, 2 and 3 months at 40° C.

### Test Methods

Three different analytical methods were employed to cover a wide aggregate and particle size range including Flowcam (2-80 microns), resonant mass measurement (0.3-4 microns) and analytical ultracentrifugation (1-400 nanometers (i.e., 0.4 microns)).

For the study described herein, pharmacopeial methods for 100% visual inspection of VPs were applied to ligelizumab formulation stored in syringes. The same twenty syringes of each packaging combination were inspected prior to storage and reinspected throughout the storage period to monitor the change in VPs and not the particles having escaped the initial visual inspection given the probabilistic nature of the method. Overall, Glass (Silicone) syringes (i.e., borosilicate glass with spray-on silicone oil lubricant) showed the highest number of VPs that increased after storage at 25° C. and at 40° C. SiO2 (PECVD) syringes (i.e., SiO2 hybrid syringe with proprietary lubricant) had lower VPs compared to Glass (Silicone) syringes but increased with storage time at 40° C. Most of the VPs detected in the drug formulations stored in SiO2 (PECVD) and Glass (Silicone) syringes were lubricant droplets. SiO2 (NONE) (i.e., SiO2 hybrid lubricant-free) had the lowest number of VPs and showed marginal increase with time at both temperatures.

Subvisible particles (SVP) are 1-100 microns in diameter and too small to be reliably detected by eye. This requires more advanced combinations of analytical methods to detect, quantify and characterize their composition, which can help determine their place of origin. For injectable drug products, SVPs are quantified according to the United States Pharmacopeia (USP) <788>, which is harmonized with European and Japanese guidance. The original goal of USP <788>guidance was to prevent blockage of capillary sized blood vessels by SVPs larger than 10 and 25 microns after intravenous drug injection. This applies predominantly to solid foreign particles made of glass or metal as an example. In contrast, SVPs in biological preparations are partly protein aggregates, which are undesirable due to a possible increased immunogenicity and/or increased formation of neutralizing antibodies.

The study evaluated SVPs larger than 2 microns by microflow imaging of protein drug formulations stored in all three syringe packaging combinations. Three different particle categories were defined based on their appearance including: 1) round silicone oil droplets 2) non-silicone oil protein-like clusters, and 3) unclassified. A large proportion of the non-silicone oil and unclassified SVPs from SiO2 (PECVD) and Glass (Silicone) syringes were clusters of smaller silicone oil SVPs. Therefore, the number of silicone oil-like SVPs are undercounted.

Flowcam was used to measure SVPs in the size range of 2 to 100 microns in ligelizumab formulation. Practically no SVPs were detected when the drug formulation was stored in SiO2 (NONE) syringes. Analytical ultracentrifugation (AUC) was used to assess monomer as well as high molecular weight (HMWS) and low molecular weight species (LMWS) in the 1 to 400 nanometer (i.e., 0.4 microns) size range.

An assessment of the break-loose force (BLF) and gliding-force (GF) was conducted on all three syringe packaging combinations. Overall, GFs were high (i.e., 12-16 N for the SiO2 packaging irrespective of storage, nearly 16 N for the spray-on siliconized glass syringe after 3 months at 40° C.) in all three syringe packaging combinations due to the high viscosity of ligelizumab formulation. Higher viscosity fluids have a higher resistance to flow compared to water and therefore will exert a greater hydrodynamic pressure on the plunger during injection. Furthermore, a larger force is required to push the plunger at the same speed down the length of the syringe barrel.

SiO2 hybrid syringes, with and without a PECVD lubricant, showed significantly lower amounts of subvisible particles (SVPs) after storage at 25° C. and 40° C.with a surfactant-containing concentrated mAb formulation compared with spray-on siliconized glass syringes. The fluid image evaluations showed that the SVPs were predominantly silicone droplets. Virtually no lubricant droplets could be measured in the SiO2 hybrid syringes without lubricant, which was also confirmed by RMM. Visible silicone droplets formed after 25° C. and 40° C. storage in the spray-on siliconized glass syringes, but significantly fewer comparatively in the PECVD lubricated SiO2 hybrid syringes. The lubricant free SiO2 hybrid syringes showed no storage-related changes, but manufacturing-related visible particles (VPs), which can be avoided by optimizing the manufacturing process (GMP process).

The silicone oil leaching shown for the SVPs and VPs resulted in a significant increase in gliding force (up to 12-15 N) compared to the initial value for the spray-on siliconized glass syringes. The SiO2 syringes did not show any storage-related changes, but stable high gliding force values for the concentrated and viscous ligelizumab formulation. Examination of the inner layers of the glass and SiO2 syringes proved the virtually complete detachment of the sprayed-on silicone layer (glass syringe) and the completely stable condition of the SiO2 layers applied via the plasma process.

This study has showed that there is a mutual interaction phenomena between protein formulation and siliconized/silicone-free syringe packaging materials with an effect on functionality and stability. Spray-on siliconized “mobile” syringes do not only pose a concern as regards potential interactions with biologics molecules, but also regarding silicone depletion due to formulation components (polysorbates and low pH) and due to protein interaction. Novel silicone-free cycloolefin polymer or glass syringes combined with novel stoppers (not involving silicone coatings) performed better according to this study for the ligelizumab formulation.

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein. The claims should not be read as limited to the described order or elements unless stated to that effect. It should be understood that various changes in form and detail may be made without departing from the scope of the appended claims.

What is claimed is:

1. A stable aqueous pharmaceutical composition comprising at least 50 mg/ml to about 150 mg/ml of an anti-IgE antibody, such as ligelizumab, about 200-300 mM trehalose, about 5-25 mM histidine, and about 0.01% to 0.05% polysorbate 20 (w/v), wherein the pH of the composition is from about 4.7 to about 5.2.

2. The aqueous pharmaceutical composition according to claim 1, wherein the anti-IgE antibody is ligelizumab.

3. The aqueous pharmaceutical composition of claim 1 or claim 2, wherein the viscosity of the composition is from about 5 to about 30 mPa-s, preferably from about 5 to about 20 mPa-s.

4. The aqueous pharmaceutical composition of any one of claims 1 to 3, wherein the pH of the composition is about 5.0.

5. The aqueous pharmaceutical composition of any one of claims 1 to 4, comprising about 0.01%-0.02% polysorbate 20 (w/v).

6. The aqueous pharmaceutical composition of claim 5, comprising about 0.02% polysorbate 20 (w/v).

7. The aqueous pharmaceutical composition of any one of claims 1 to 6, comprising about 10-25 mM histidine buffer.

8. The aqueous pharmaceutical composition of claim 7, comprising about 20 mM histidine.

9. The aqueous pharmaceutical composition of any one of claims 1 to 8, comprising about 250 -270 mM trehalose.

10. The aqueous pharmaceutical composition of claim 9, comprising about 250 mM trehalose.

11. The aqueous pharmaceutical composition of any one of claims 1 to 10, comprising between about 60 mg/ml and about 120 mg/ml of the anti-IgE antibody.

12. The aqueous pharmaceutical composition of claim 11, comprising about 120 mg/ml of the anti-IgE antibody.

13. A stable aqueous pharmaceutical comprising about 120 mg of ligelizumab, about 250 mM trehalose, about 20 mM histidine, and about 0.02% polysorbate 20 (w/v), wherein the pH of the composition is about 5.0.

14. The aqueous pharmaceutical composition of any of one of claims 1 to 13, wherein said composition is stable for at least 18 months at 2-8° C.

15. An aqueous pharmaceutical composition of any one of claims 1-14 for use in delivering the anti-IgE antibody to a subject in need thereof, comprising a step of administering the aqueous pharmaceutical composition to the subject.

16. An aqueous pharmaceutical composition of any one of claims 1-14 for use in treating an allergy that is mediated by IgE, comprising administering the aqueous pharmaceutical composition to a subject in need thereof.

17. A dosage form comprising the aqueous pharmaceutical composition of any one of claims 1-14.

18. A delivery device comprising the aqueous pharmaceutical composition of any one of claims 1-14.

19. The delivery device of claim 18, which is a pre-filled syringe.

20. The delivery device of claim 18, which is an automated disposable injection device, such as an auto-injector.
